(19) United States
(12) Patent Application Publication
(10) Pub. No.: US 2025/0128230 A1
(43) Pub. Date: Apr. 24, 2025

(54) Nanoengineered Hydrogels and Uses Thereof

(71) Applicants: Kaivalya Deo, Berkeley, CA (US); Akhilesh Gaharwar, Cypress, TX (US)

(72) Inventors: Kaivalya Deo, Berkeley, CA (US); Akhilesh Gaharwar, Cypress, TX (US)

(73) Assignee: The Texas A&M University System, College Station, TX (US)

(21) Appl. No.: 18/943,159

(22) Filed: Nov. 11, 2024

Related U.S. Application Data
(63) Continuation of application No. PCT/US2023/066868, filed on May 11, 2023.
(60) Provisional application No. 63/340,849, filed on May 11, 2022.

(51) Int. Cl. B01J 13/00; A61B 5/00; A61B 5/11; A61N 1/04; B82Y 5/00; B82Y 40/00

(52) U.S. Cl. CPC B01J 13/0065; B82Y 5/00; B82Y 40/00; A61B 5/11; A61B 5/4836; A61B 5/6861; A61B 2562/166; A61N 1/04

(57) ABSTRACT

Provided herein are nanoengineered biomaterial inks used to 3D print a biocompatible, flexible electronic device, for example, a hydrogel constructs a biocompatible, flexible electronic device or 3D printed wearable electronic devices. The biocompatible, flexible electronic device may be a crosslinked gelatin-SH-2D-MoS₂ nanoassembly. Also provided are methods for nanoengineering a biomaterial ink and for determining a treatment for a subject in need thereof utilizing the biocompatible, flexible electronic device.

### CROSS-REFERENCE TO RELATED APPLICATIONS

This application is a continuation under 35 U.S.C. § 120 and 35 U.S.C. § 365(c) of pending international application PCT/US2023/066868, filed May 11, 2023, which claims benefit of priority under 35 U.S.C. § 119(e) of provisional application U.S. Ser. No. 63/340,849, filed May 11, 2022, now abandoned, the entireties of which are hereby incorporated by reference.

### STATEMENT REGARDING FEDERALLY SPONSORED RESEARCH

This invention was made with government support under DP2 EB026265 awarded by the National Institutes of Health and CBET 1705852 awarded by the National Science Foundation Award. The government has certain rights in the invention.

### BACKGROUND OF THE INVENTION

### Field of the Invention

The present invention relates to the fields of tunable hydrogels, bioelectronics and 3D printing. More specifically the present invention relates to 3D printable nanoengineered biomaterial inks for use in printing flexible bioelectronics.

### Description of the Related Art

Flexible electronics with biointerfaces have diverse applications in the field of biomedical research (1,2) including strain/pressure sensors (2,3), optoelectronics (4), implantable bioelectronics (5) and thermal/electrical actuators as therapeutic modalities (6). Regardless of the progress in flexible bioelectronics, the bulk of devices being fabricated still utilize materials which are mechanically and compositionally disparate from anatomical tissues (7).

Traditional systems lack conformity with the human body thus making wearable devices obtrusive, susceptible to noise and limited to specific regions of body. Many such devices are fabricated from components that comprise inorganic electronics employing elements like Si, W, Au, Pt etc. which may result in rigid and non-compliant devices (10 MPa-100 MPa) that are incompatible when integrating with soft tissue structures (8,9). However, most of the soft tissues in the body possess a Young's modulus approximately three orders lower in magnitude (˜kPa range) and possess significant water content (10). Therefore, the present applications of current biosensors, wearable electronics and implantable healthcare are limited (11). Hence, in recent years there has been heightened interest in developing flexible and compressible electronic biointerfaces which integrate external hardware with human body (12,13).

Two-dimensional (2D) nanomaterials have been used to design conductive hydrogels owing to their exceptionally large surface to volume ratio and high electron density (14,15). 2D transition metal dichalcogenides (TMD), like molybdenum disulfide (MoS₂) are an emerging class of materials which recently have gained exceptional attention because of their layered, atomically thin, well-defined structure which imparts interesting physico-chemical properties in comparison to their 3D counterparts (16-18). Molybdenum disulfide have been widely applied in the field of conductive electronics (19), batteries (20), catalysis (21), sensing (22), drug delivery (23) and cancer treatments (24). Despite intriguing properties and favorable biocompatibility, MoS₂ have not been fully utilized in bioengineering due to limited binding sites for conceiving tissue engineering scaffold.

There has also been significant interest in designing conductive inks because of the potential to produce complex 2D or 3D flexible bioelectronics. These malleable electronic biointerfaces fabricated through 3D printing are customizable, cost-effective, and robust. Contrary to other traditional microfabrication approaches which leads to planar designs, printing permits conception of complex 3D circuits (25). The conductive inks currently being employed in designing printed electronics usually utilize volatile solution mixtures of semi-conducting or conducting materials such as graphene, metallic nanorods, conductive polymer nanoparticles and poly(3,4-ethylenedioxythiophene) (PEDOT) (26). However, these inks lack certain necessary mechanical properties and may be cytotoxic or lack crucial biological cues.

Thus, there is a need and desire in the art to develop advanced conductive and bioactive inks for engineering flexible bioelectronics (27). The present invention fulfils this longstanding need and desire in the art.

### SUMMARY OF THE INVENTION

The present invention is directed to a hydrogel construct. The hydrogel construct comprises a 2D MoS₂ nanoassembly crosslinked with a natural polymer.

The present invention also is directed to a method for nanoengineering a biomaterial ink. In the method synthesizing a defect dense 2D MoS₂ nanoassembly is synthesized and a gelatin is thiolated to produce a thiolated gelatin. The 2D MoS₂ nanoassembly is cross-linked with the thiolated gelatin, thereby nanoengineering the biomaterial ink. The present invention is directed to a related method further comprising 3D printing a biocompatible, flexible electronic device from the biomaterial ink.

The present invention is directed further to a biocompatible, flexible electronic device nanoengineered by the method described herein.

The present invention is directed further still to a 3D printed wearable electronic device configured to monitor motion of a subject. The 3D printed wearable electronic device is a 2D-MoS₂ nanoassembly comprising a plurality of sulfur vacancies therein and a gelatin comprising a plurality of thiols attached thereto. Each of the plurality of thiols are crosslinked within one of the plurality of sulfur vacancies.

The present invention is directed further still to a method for determining a treatment for a subject in need thereof. In the method the wearable electronic device described herein is placed on or in the subject. Dynamic parameters of the subject are monitored via the wearable electronic device and a treatment for the subject is selected based on results of the monitoring step. The present invention is directed to a related method where the wearable electronic device is an electroceutical and the method further comprises delivering the treatment to the subject.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1L illustrate the synthesis and fabrication of nanoengineered hydrogel bioinks through defect-driven gelation. FIG. 1A: SEM image of MoS₂ (1:1, 1:2, 1:4, 1:6) nanoassembly agglomerates exhibiting the nano-flower morphology in all ratios and displaying spherically arranged rippled flakes. FIG. 1B: TEM and FIG. 1C: AFM imaging of dispersed MoS₂ nanoassemblies confirming the rippled architecture. FIG. 1D: X-ray diffraction pattern obtained for MoS₂ nanoassemblies affirming the 2H phase of MoS₂ lattice. FIG. 1E: Shear-thinning behavior of precursor solutions. 5 wt % thiolated gelatin (Gel-SH) solution exhibits Newtonian behavior whereas with addition of MoS₂ nanoassemblies there is increase in viscosity of precursor solutions and they exhibit shear-thinning behavior. Power law model was fitted to establish consistency coefficient and power law index. FIG. 1F: Gelation kinetics of Gel-SH with and without MoS₂ validating defect-driven gelation between Gel-SH and MoS₂ nanoassemblies. Additionally, photographs show formation of crosslinked hydrogels which is even stable at higher temperatures. FIG. 1G: Stress sweep illustrating interactions between hydrogel precursors under oscillatory shear stress through cross-over of storage (G′) and loss (G″) modulus. FIG. 1H-1J: Oscillatory stress sweeps of 0.5%, 1% & 1.5% MoS₂/Gel-SH precursors exhibit successive higher yield stresses with increasing concentration of MoS₂ nanoassemblies indicating enhanced polymer/nanoassembly interaction. FIG. 1K: Peak hold test simulating flow during extrusion/printing through an extruder tip and demonstrating significant recovery post extrusion indicating potential applications in 3D printing. FIG. 1L: Creep and recovery curves for Gel-SH and MoS₂/Gel-SH solutions. Gel-SH is plotted on right y-axis (as indicated by arrow) and does not demonstrate recovery whereas 1% & 1.5% MoS₂/Gel-SH solutions are plotted on left y-axis and demonstrate creep-recovery behavior. Burger model was fitted on the creep region to establish instantaneous compliance (J₀), retarded compliance (J₁), Newtonian viscosity (η) and retardation time (T).

FIGS. 2A-2D show 3D printability of nanoengineered ink. The STL file and actual 3D printed structures of an oval band (FIG. 2A), rectangular lines (FIG. 2B), a cross hatch grid (FIG. 2C) and a hollow cylinder (FIG. 2D) are compared. The ability to print shapes with diverse sizes, dimensions and height/thickness demonstrates versatility of the nanoengineered ink.

FIGS. 3A-3M show mechanical and chemical characterization of crosslinked MoS₂/Gel-SH hydrogels investigating hydrogel architecture, conforming defect-driven gelation and examining its elastomeric attributes. FIG. 3A: SEM and Energy-dispersive X-ray spectroscopy (EDS) images of a transverse cross-section of hydrogels (0.5% & 2% MoS₂/Gel-SH) exhibiting porous, interconnected network with presence of Molybdenum and Sulphur. FIG. 3B: EDS spectra and mapping confirmed the presence of Mo and S in the crosslinked hydrogel network. FIG. 3C: Raman spectra of Gel-SH control, 0.5% & 2% MoS₂/Gel-SH gels. The peak for C—S stretch at 760 cm⁻¹ was shifted and attenuated in MoS₂ gels. Additionally, peak intensities of thiol vibrations at 1099 and 2330 cm⁻¹ reduced substantially with the addition of MoS₂ nanoassemblies. FIG. 3D: XPS of hydrogels exhibits the change in binding energy of carbon (C 1s) peak and appearance of an additional peak in crosslinked sample due to C—S—Mo formation. FIG. 3E: XPS spectra of Molybdenum shows emergence of additional peak belonging to MoS₂—GeISH. FIG. 3F: Demonstration of high mechanical flexibility and elastomeric nature of crosslinked hydrogels with more than 80% compression. FIG. 3G: Uniaxial cyclic compression demonstrates successful compression and recovery of hydrogels for successive strain cycles of 20%, 40%, 60% and 80% strain. FIG. 3H: Compressive modulus of hydrogels illustrates drastic increase in modulus values from 4 kPa of thermally gelled Gel-SH controls to 55 kPa of covalently crosslinked 1.5% MoS₂/Gel-SH hydrogels. FIGS. 3I-3J: Plot of toughness and maximum stress through cyclic compressive testing illustrates increase in values of both toughness and maximum stress with increasing concentration of MoS₂ nanoassemblies due to covalent crosslinking and reinforcement in MoS₂/Gel-SH gels leading to formation of robust hydrogel networks. Statistical analysis was performed using one-way ANOVA including posthoc Tukey's test with *p<0.05, **p<0.01. FIG. 3K: Elongation tensile testing of nanocomposite hydrogels and examining their tensile behaviors through stress-strain curves. FIG. 3L: Tensile modulus of 0.5% and 2% MoS₂/Gel-SH hydrogels. Statistical analysis was performed using one-way ANOVA including posthoc Tukey's test with *p<0.05, **p<0.01. FIG. 3M: Comparing the tensile toughness of the hydrogels. Statistical analysis was performed using one-way ANOVA including posthoc Tukey's test with **p<0.01.

FIGS. 4A-4J shows the electrochemical characterization, in vitro and in vivo cytocompatibility of MoS₂/Gel-SH hydrogels. FIG. 4A: Electrical impedance spectroscopy (EIS) was performed on the hydrogels. The full spectrum Nyquist plot demonstrates variation in real and imaginary impedance components of hydrogels with varied concentration of MoS₂ nanoassemblies. Bode plots of MoS₂/Gel-SH networks between a frequency range of 0.01 Hz and 1 MHz demonstrate variation of impedance magnitude and phase with frequency. The EIS data was further analyzed and interpreted through Randles Equivalent Circuit model which established values of membrane resistance (R_{M}), charge-transfer resistance (R_{CT}) and constant phase element (Q_{DL}). FIG. 4B: Inclusion of MoS₂ nanoassemblies lead to drop in the membrane resistance of the system. FIG. 4C: The charge-transfer resistance of MoS₂/Gel-SH hydrogels were significantly lower compared to Gel-SH control confirming electroactivity of MoS₂ nanoassemblies. FIG. 4D: The increasing values of constant phase element with inclusion of MoS₂ nanoassemblies demonstrate non-ideal double layer capacitive behavior of the hydrogels. FIG. 4E: Cyclic Voltammetry (CV) measurements of the MoS₂/Gel-SH hydrogels exhibit pseudo-capacitive charge storage behavior at higher concentrations of MoS₂ nanoassemblies. FIG. 4F: Multiple scan rates cycles voltammetry (MSRCVs) of 10% MoS₂/Gel-SH hydrogels demonstrated maintenance of the pseudo-capacitive response at different scan rates. FIG. 4G: Picogreen viability assay illustrates continued cell viability over 1, 3 and 7 days in presence of MoS₂/Gel-SH hydrogels. FIG. 4H: Live/Dead imaging of cells seeded over the MoS₂/Gel-SH hydrogels after a period of 3 days demonstrating high cell viability. FIG. 4I: Cellular cytoskeletal staining demonstrates successful attachment and spreading of cells over various MoS₂/Gel-SH hydrogels. FIG. 4J: Subcutaneous implantation demonstrates in-vivo biocompatibility of the MoS₂/Gel-SH hydrogels. Statistical analysis was performed using one-way ANOVA including posthoc Tukey's test with *p<0.05, **p<0.01, ***p<0.001 ****p<0.0001.

FIGS. 5A-5G show a strain sensing and dynamic motion capture analysis. FIG. 5A: The elastomeric MoS₂/Gel-SH hydrogel was able to respond successive compression cycles (1-5 cycles) through relative change in resistance values (ΔR/R₀) as determined via electromechanical measurements. FIG. 5B: Electromechanical characteristics of MoS₂/Gel-SH hydrogels are further evaluated with respect to applied force and strain. A linear relationship between the change in resistance (ΔR/R₀) and external force/strain demonstrates a wide scale of detection capabilities. FIG. 5C: The wearable hydrogel device was successively bent at 45° and 30° from both ends and a corresponding resistance change was recorded for each bending motion cycle. FIG. 5D: Bending the hydrogel device from both ends at about 90° exhibited a significantly higher change in resistance. FIG. 5E: Various degrees of deformation were co-related to equivalent resistance change values. FIG. 5F: The device was bent at two different motion speeds and its equivalent changes in resistance rates were reported indicating measurement sensitivity of the device. FIG. 5G: Twisting action of the device at various deformation angles recorded a specific resistance change signature for the motion. Statistical analysis was performed using one-way ANOVA including posthoc Tukey's test with *p<0.05, **p<0.01, ***p<0.001 ****p<0.0001.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the term “including” is used herein to mean “including, but not limited to”. “Including” and “including, but not limited to” are used interchangeably.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the term “subject” refers to a mammal, preferably a human, on whom is placed the hydrogel construct, biocompatible, flexible electronic device or 3D printed wearable electronic device presented herein.

In one embodiment of the present invention there is provided a hydrogel construct, comprising a 2D MoS₂ nanoassembly crosslinked with a natural polymer. In this embodiment the hydrogel construct is a crosslinked gelatin-SH-2D-MoS₂ nanoassembly.

In this embodiment the 2D MoS₂ nanoassembly may be defect dense. Particularly, the 2D MoS₂ nanoassembly may comprise a defect ratio of about 1:0 to about 1:10 molybdenum:sulfur. In an aspect thereof the 2D MoS₂ nanoassembly may comprise a defect ratio of about 1:3 to about 1:10. A non-limiting example of the defect ratio is 1:6 molybdenum:sulfur. Also in this embodiment the natural polymer may be gelatin, hyaluronic acid, pectin, collagen, carrageenan, chitosan, cellulose, silk fibroin, keratin, alginate, elastin, glucomannan, fibrinogen, gelatin, lignin, starch, xanthan gum, zein, poly-γ-glutamic acid (γ-PGA) or pullulan. In this embodiment the natural polymer is thiolated. In addition the hydrogel construct comprises a crosslinked gelatin-SH-2D-MoS₂ nanoassembly. Furthermore the hydrogel construct is a 3D printable, biocompatible biomaterial ink.

In another embodiment of the present invention there is provided a method for nanoengineering a biomaterial ink, comprising synthesizing a defect dense 2D MoS₂ nanoassembly; thiolating a gelatin to produce a thiolated gelatin; and crosslinking the 2D MoS₂ nanoassembly with the thiolated gelatin, thereby nanoengineering the biomaterial ink. Further to this embodiment the method comprises 3D printing a biocompatible, flexible electronic device from the biomaterial ink.

In one aspect of both embodiments the synthesizing step may comprise constructing the 2D MoS₂ nanoassembly with a dense defect ratio of about 1:2 to about 1:10 molybdenum:sulfur. Particularly, the dense defect ratio is 1:6 molybdenum:sulfur. In another aspect of both embodiments the crosslinking step may comprise conjugating thiols on the thiolated gelatin in sulfur vacancies within the 2D MoS₂ nanoassembly without external stimuli or an initiator.

In yet another embodiment of the present invention there is provided a biocompatible, flexible electronic device nanoengineered by the method as described supra. In this embodiment the 2D MoS₂ nanoassemblies are electronically conductive. In aspects of this embodiment the biocompatible, flexible electronic device may comprise an electroceutical, a wearable device, an ingestible device, an implantable device, an energy storage device, or an a light actuating conductive device.

In yet another embodiment of the present invention there is provided a 3D printed wearable electronic device configured to monitor motion of a subject, comprising a 2D-MoS₂ nanoassembly comprising a plurality of sulfur vacancies therein; and a gelatin comprising a plurality of thiols attached thereto, each of the plurality of thiols crosslinked within one of the plurality of sulfur vacancies.

In this embodiment the 2D-MoS₂ nanoassembly may comprise a defect ratio of at least 1:2 molybdenum:sulfur. Particularly, the 2D-MoS₂ nanoassembly may comprise a defect ratio of about 1:2 to about 1:10 molybdenum:sulfur. Also in this embodiment the 3D printed wearable electronic device may be a flexible, biocompatible hydrogel construct. In an aspect thereof the flexible, biocompatible hydrogel construct may be an electroceutical. In addition the 3D printed wearable electronic device may be an ingestible device. Furthermore the 3D printed wearable electronic device may comprise hybrid conductivity, charge storage or tissue-like mechanical properties. In an aspect of this embodiment

In yet another embodiment of the present invention there is provided a method for determining a treatment for a subject in need thereof, comprising placing the wearable electronic device, as described supra, on or in the subject; monitoring at least one dynamic parameter of the subject via the wearable electronic device; and selecting a treatment for the subject based on results of the monitoring step. Further to this method the wearable electronic device is an electroceutical, the method comprising delivering the treatment to the subject. In both embodiments the dynamic parameter may be motion, temperature, humidity, electric current, flow, strain, or pressure or a combination thereof.

The present invention enables the nanoengineering of highly elastomeric, shear-thinning hydrogel constructs with tunable electronic, ionic and capacitive response. by combining defect-rich MoS₂ nano-assemblies with thiolated gelatin (Gel-SH). The hydrogel precursors demonstrate tunable rheological characteristics, enabling 3D printing of a complex pattern which provides opportunities for its use in development of shear-thinning nanoengineered biomaterial ink. Further controlling MoS₂ concentration, the covalent network exhibits hybrid conductivity with a tailored pseudocapacitive response due to faradic and capacitive currents. The engineered Gel-MoS₂ hydrogel construct exhibit suitable in vitro and in vivo biocompatibility. The constructs further demonstrate extraordinary motion sensing capabilities in response to mechanical strain. The 3D printed hydrogels were utilized for engineering flexible wearable or ingestible electronic devices accurately monitoring various dynamic parameters or dynamic strain states, and are utilizable as implantable bioelectronics for monitoring human motion and other dynamic parameters. Non-limiting examples of dynamic parameters are temperature, humidity, electric current, flow, strain, or pressure or combinations thereof.

The following examples are given for the purpose of illustrating various embodiments of the invention and are not meant to limit the present invention in any fashion.

### Example 1

### Experimental Methods

### Synthesis of Defect-Rich MoS₂ Nanoflower Assemblies

The synthesis of 1:6 defect-rich MoS₂ nanoflower assemblies was achieved by dissolving 1 mmol of hexaammonium heptamolybdate tetrahydrate, (NH₄)₆·Mo₇O₂₄·4H₂O and 42 mmol of Thiourea, NH₂CSNH₂ (from Alfa Aesar) in 50 mL of deionized (DI) water. To synthesize other defect ratios of MoS₂ nanoassemblies (1:1, 1:2, and 1:4) the reagent molar ratios were varied accordingly, (e.g. 1:1 MoS₂ was synthesized using 1 mmol of (NH₄)₆·Mo₇O₂₄·4H₂O and 7 mmol of thiourea). This solution was vortexed in a falcon tube to dissolve all constituents and transferred to a hydrothermal vessel. The vessel was subsequently autoclaved at 220° C. for 18 h. The solution was then allowed to cool down to room temperature and the MoS₂ nanoflower assemblies were washed alternatively several times with ethanol and DI water to remove unreacted impurities. To purify them further, the nanoassemblies were transferred into a dialysis bag and dialyzed for three days, with water being changed every 6 h.

### Synthesis of Thiolated Gelatin

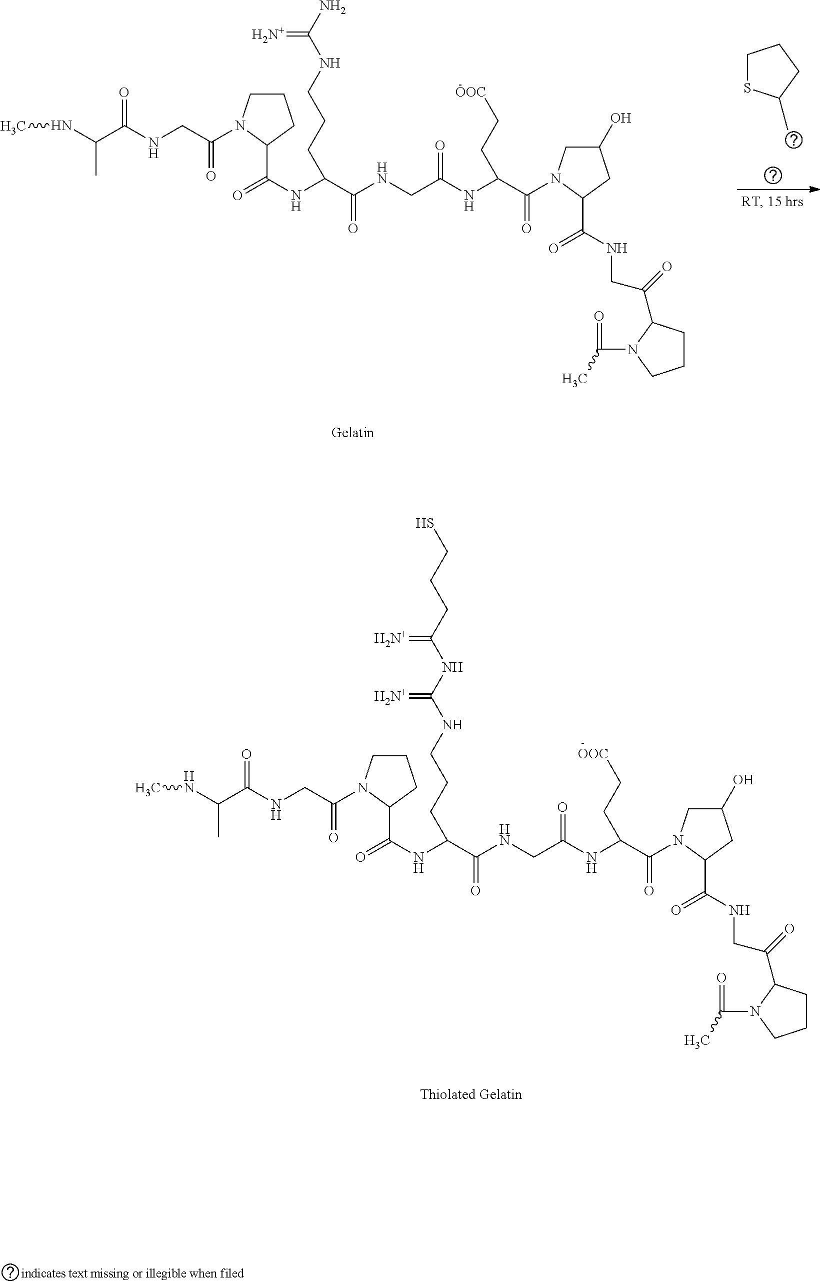

Thiolated gelatin was synthesized by covalently modifying the primary amine groups of gelatin through addition of sulfhydryl groups (36). 2 g of gelatin and 200 mg of 2-iminothiolane hydrochloride (Traut's reagent) was dissolved in 200 mL of ax phosphate buffer solution (PBS). The mixture was left stirring for 15 h at room temperature. To remove any unreacted iminothiolane hydrochloride, the mixture was dialyzed sequentially against 5 mM HCl and then 1 mM HCl solution for 24 h each. The resultant thiolated gelatin solution was lyophilized and stored at 80° C. for further use.

### Thiolation of Gelatin and Degree of Thiolation Determination

The degree of thiolation was determined by dissolving thiolated gelatin and control gelatin in 0.1 M sodium phosphate buffer (pH 8.0) with 1 mM EDTA. Subsequently, 100 μL of Ellman's reagent (4 mg/mL), 500 μL of sample solutions and 5 mL of buffer were mixed. The mixture was kept for 15 mins at room temperature for the calorimetric reaction to proceed after which absorbance for all the solutions was measured at 412 nm by spectrophotometer. The degree of thiolation was expressed in terms of millimoles of free sulfhydryl groups per gram of gelatin and was calculated by extrapolating the obtained results to that of the standard curve of cysteine. The degree of thiolation was calculated using the mentioned procedure and was found out to be 22.4 mM/g of gelatin. Gelatin thiolation was also corroborated by performing Fourier transform infrared spectroscopy (ATR-FTIR). FTIR was performed on gelatin and thiolated gelatin with a Bruker Vector 22 FTIR spectrophotometer (PIKE Technologies). Primary/secondary amine peaks at 3010 and 3130 cm⁻¹ were found to be attenuated in Gel-SH spectra compared to gelatin control confirming thiolation.

### Shear-Thinning Conductive Ink and Chemically Crosslinked Hydrogels

The conductive ink and covalently crosslinked hydrogels were made by preparing a 5 wt % of thiolated gelatin mixture in a dispersion of 1:6 defect-rich MoS₂ nanoflower assemblies in DI water. The concentration of thiolated gelatin was selected to provide sufficient viscosity, extrudability and shear-thinning behavior for enabling 3D printing. The concentration of MoS₂ assemblies was varied from 0.5% to 10% depending on the experimental study. This prepolymer mixture was probe sonicated for 1 min and subsequently stirred to obtain a homogeneous mixture. The precursor mixture is left to gel for up to 5 h to obtain hydrogels with appropriate mechanical stability. The mixture can further be injected into a mold and allowed to gel to make hydrogels of certain shape and dimensions. The crosslinked hydrogels are removed from the mold and soaked in PBS/DI water to remove any unreacted moieties.

Characterization of MoS₂ Nanoassemblies and MoS₂/Gel-SH Hydrogels

For morphological characterization and imaging of the MoS₂ nanoassemblies and freeze-dried crosslinked hydrogels, FEI Quanta 600 field emission-scanning electron microscopy (FE-SEM) was used. The samples were coated through Pt/Pd plasma coating to the thickness of 10 mm to improve their conductivity. They were then imaged under FE-SEM where secondary electron (SE) mode with an operational voltage of 15 keV was utilized. For elemental mapping in the hydrogels to discern distribution of molybdenum and sulfur, Energy dispersive X-ray spectra (EDS) detector was utilized by selecting particular regions in the hydrogels and, in the selected areas, the presence of elements was mapped by documenting the binding energy spectra. The MoS₂ nanoassemblies were also imaged through transmission electron microscopy (TEM), for which the MoS₂ samples were dispersed, drop-casted and air-dried on a copper grid (from Ted Pella Inc.). The TEM imaging was accomplished through JEOL 2010 which was operated at 200 keV. The crystallographic characterization of the defect-rich MoS₂ nanoflower assemblies was accomplished through X-ray powder diffraction (XRD) (Bruker D8 advanced). The XRD was performed using a copper source (wavelength ˜1.5406 Å, an operating current of 40 kV and 25 mA) and data was recorded from 5 to 70 degrees. The X-ray optics was the standard Bragg-Brentano para-focusing mode with the X-ray diverging from a DS slit (1 mm) at the tube to strike the sample and then converging at a position sensitive X-ray Detector (Lynx-Eye, Bruker-AXS). The software suit for data collection and evaluation is Windows-based. Data collection is automated COMMANDER program by employing a DQL file. Data is analyzed by the program EVA. The indexing of the acquired peaks was done using JCPDS card number 73-1508. The crystallite size (D) was calculated from the characteristic peak (002) by using the Scherrer equation:

$$D = \frac{λ K}{β cos θ}$$

D=^{A}B where A is X-ray wavelength (1.5406 Å), K is the shape factor (˜0.94), 6 is the Bragg angle and p is line broadening at full width half maxima (FWHM).

Raman and XPS spectroscopy analysis

Raman spectroscopy was performed to validate the phase composition of the chemically crosslinked hydrogels. The hydrogels were placed on glass slide and excited by 532 nm green laser to acquire Raman spectra (LabRam HR confocal Raman microscope, Horiba Inc. Japan) between 250 to 2850 cm¹. Further the phase composition of the MoS₂ nanoassemblies and their chemical conjugation to the polymer chains was validated using X-ray photoelectron spectroscopy (XPS) (Omicron XPS system with Argus detector). The binding energies (B.E.) for carbon (1s), sulphur (2p) and molybdenum (3d) was recorded. The data was further deconvoluted and processed by CasaXPS multiple peak fit software and indexed using standard library.

### Rheology

The gelation kinetics, viscoelasticity characterization, creep-recovery behavior and stress sweep rheological testing was carried out with a stress-controlled rheometer (DHR-2 discovery hybrid rheometer, TA Instruments, New Castle, Delaware) using a 20 mm parallel plate geometry at a gap of 0.2 mm in conjunction with a solvent trap. Creep experiments were conducted by applying 50 Pa stress for 10 mins followed by 15 min of recovery (no applied stress). Data from creep experiments were evaluated by attempted fitting of the creep region with the Burger model:

$${J}_{c} ( t ) = {J}_{0} + {J}_{1} [ 1 + exp ( - \frac{t}{τ} ) ] + t / η$$

where J_{c}(t) is the measured compliance of samples at time t. J₀ is the instantaneous compliance and J₁ is the retarded compliance corresponding to Maxwell and Kelvin-Voigt elements, respectively. η is the Newtonian viscosity and T is the retardation time³⁷. Stress sweep and stress relaxation was also performed on crosslinked hydrogel samples (˜7.5 mm×2.5 mm). Stress relaxation was performed at incremental strains of 5%, 10%, 15% and 20% for all the hydrogel samples. All rheological experiments were performed at room temperature otherwise mentioned otherwise.

### Uniaxial Compression Testing

To determine the compressive modulus of the hydrogels, cyclic compression testing was performed using ADMET eXpert 7600 system (ADMET, Inc., Norwood, Massachusetts) with attached load cell of 25 lb. The testing was performed at a strain rate of 1 mm/min on cylindrical hydrogel samples (˜7.5 mm×2.5 mm) with variable strains of 20%, 40%, 60% and 80% to demonstrate network resilience, while compressive modulus was calculated corresponding to 10%-20% strain region of the engineering stress-strain curve (dividing force with original cross-section area).

### Elongation Tensile Testing

Tensile Young's modulus of the hydrogels was determined by making rectangular hydrogels samples with dimensions 25×5×1 mm (length×width×thickness) and performing tensile testing using Instron 5944 Universal Testing System equipped with tensile clamps. Specimens were strained at a rate of 1 mm/min. From the resulting stress-strain curves, Young's modulus (E) and toughness was calculated.

### Swelling and Degradation

Hydrogels were synthesized with 5 wt % of thiolated gelatin and different concentrations of MoS₂ nanoassemblies (0.5, 1, 1.5, 2 wt %). Control samples were prepared by thermally gelling 5 wt % of thiolated gelatin at 37° C. All the prepared samples were weighed and transferred to a 24 well plate with 1.5 ml of either PBS (pH 7.4) or 5 mg/mL collagenase IV solution (reconstituted in PBS) for hydrolytic or enzymatic degradation studies, respectively. The hydrogel samples were incubated at 27° C. and weight measurements were taken at 0, 72, 168 and 504 hours for hydrolytic degradation and 0, 12, 36 and 504 hours for enzymatic degradation. After each measurement, fresh PBS or collagenase solution was replenished into each respective well. Swelling ratios for the hydrogels were determined using the following relation:

$${Q}_{m} = \frac{Weight ( t > 0 )}{Weight ( t = 0 )} .$$

### 3D Printing

The shapes to be 3D printed were generated in Solidworks and converted to STL files. The STL files were transferred into Slic3r to customize printing variables and converted into G-code instructions for printer. 3D printing was performed by a modified Anet A8 printer with a screw extruder-based printing head. Repetier-Host program was used to interface with the 3D printer. 3D printing was executed with a 410 μm tapered tip attached to the extruder. Printing speed was 5 mm/see, layer height during printing was kept at 200 μm and layer width was set at 300 μm. Using these settings following shapes were printed. An oval circular band with length of 50 mm, width 12 mm and thickness of band 2 mm. Rectangular curved lines with length 20 mm and width of 5 mm. A square crosshatch grid with side length of 18 mm. A hollow cylinder with outer diameter of 10 mm, internal diameter of 9 mm and height of 12 mm.

### Electrochemical Characterization

The electrical and electrochemical properties of MoS₂/Gel-SH hydrogels (MoS₂=0, 0.5, 2, 5 and 10 wt %) were measured via two-electrode electrical impedance spectroscopy (EIS) and three-electrode cyclic voltammetry (CV). The hydrated hydrogel disks, previously equilibrated in 0.1 M PBS 7.4 at RT, were sandwiched between a pair of circular (φ=8 mm), gold-coated, polyamide electrodes that were cleaned in methanol. EIS measurements were done at an interrogation voltage of 20 mV p-to-p and over the frequency range of 0.01 Hz-10 MHz at RT. The data were then modeled with a simplified Randles Equivalent Circuit using ZSimpWin 3.60 (EChem Software, Ann Arbor, MI). Multiple scan rate CV were obtained with the MoS₂—SH-Gel cast onto a single, gold-coated polyamide electrode that served as the working electrode, a platinum counter electrode, and an Ag/AgCl as reference electrode. Measurements were made using the VersaSTAT in 0.1 M PBS 7.4 at RT over the range −0.2 to 0.8 V at scan rates of 50-1000 mV/s. Four-point probe conductivity was measured on hydrogel pellets using a Keithley multimeter (Model 2010, Keithley Instruments, Inc., Cleveland, OH).

### Strain Sensing and Resistance-Change Measurements

The electromechanical measurements of the hydrogels were performed by a Mark-10 Force Gauge Series 5 and ESM 303 (Mark-10 Crop., Copiague, New York) and NI USB-4065 digital multimeter (NI Corp., Austin, Texas).

### In Vitro Cytocompatibility

All in vitro experiments were performed with human mesenchymal stem cells (hMSCs) passage 4, obtained from Lonza. hMSCs were cultured in α-modified minimal essential media (αMEM, Hyclone), 16.5% FBS (Atlanta Biological) and 1% penicillin/streptomycin (100 U/100 μg/mL; Gibco), supplemented with 50 μM ascorbic acid (BDH Chemicals) and 10 mM β-glycerophosphate (Sigma-Aldrich) at 37° C. with 5% CO₂. Metabolic activity was measured using PicoGreen dsDNA quantitation assay (ThermoFisher Scientific) using manufacturer's protocol. Briefly, the hydrogels with various concentrations of MoS₂ nanoassemblies were plated in a 96-round bottom well plate. The cells were seeded and cultured on top of the hydrogels. At designated time points 100 μl PicoGreen reagent was added to each well and read at excitation and emission wavelengths of 480/520 nm on a spectrophotometer. For morphological characterization and cytoskeletal staining, the cells were seeded on the hydrogels similar to as for viability assessment. After 72 h the cells were fixed and stained with rhodamine-phalloidin (Alexa-Fluor 594; Invitrogen) and 4′,6-diamidino-2-phenylindole (DAPI; Sigma) to visualize F-actin filaments and cell nuclei, respectively. For live/dead imaging, similar to previous procedure the cells were seeded on the hydrogels and after 72 h a prepared Live/Dead assay reagent of Calcein AM and Ethidium Homodimer (Santa Cruz Biotechnology, Inc., USA) was added to the cells and incubated for 30 min at 37° C. For both actin/dapi and live/dead imaging, the samples were then washed with PBS and imaged under epifluorescence microscope (TE2000-S, Nikon, USA).

### In Vivo Subcutaneous Implantation

Male C57BL/6J mice (n=4; ˜250 g, Charles River, MA, USA) were housed in the Small Animal Facility (Texas A&M University). All animal use procedures were approved by the Institutional Animal Care and Use Committee of Texas A&M University (IACUC No. 0133) and were performed in accordance with the NIH “Guide for the Care and Use of Laboratory Animals”. On day 1, animals were chosen at random and anesthetized by administration of isoflurane (Isoflurane at 3-4% (v/v) for induction of anesthesia and maintenance at 2.0% (v/v) in 02). For this purpose, the conscious animals were placed in a sterile chamber, into which the gas mix was slowly fed. Once under anesthesia, incision sites were sheared and cleaned with Chlorhexidine. All animal surgeries were carried out in a sanitized suite where the metal work surfaces were covered with sterilized cotton towels. While working on them, animals are kept on a sterile warm mat maintained at 34° C. Sterile, crosslinked GeIMA and 0.5% MoS₂/Gel-SH hydrogel discs with thickness of 2 mm and a diameter of 6 mm were implanted in a dorsal pocket with not more than one sample from a group implanted per animal according to a randomized scheme. The wound was closed using wound clips. The animal was solitarily housed for one day postoperatively and afterward in same-sex groups at the Small Animal Facility. On day 7, GeIMA and MoS₂/Gel-SH groups were placed in separate enclosures and euthanized using CO₂ inhalation. Post euthanasia, explants containing hydrogel discs were surgically extracted from the dorsal pockets and immediately washed with 1× sterile PBS. The discs were then individually fixed in freshly prepared 4% paraformaldehyde for 3 hours and transferred to 70% ethanol for transport. The fixed explanted tissue groups were sent to the Histology Research Unit at Texas A&M university for sectioning. Tissue samples were sectioned into 15 μm thick sections for histological analysis. Tissue sections were deparaffinized using xylene and serially rehydrated using 100%, 95%, 80%, and 70% ethanol. Slides for each group were then incubated in hematoxylin for 5 minutes and washed under tap water. The slides were placed in Scott's bluing solution till blue stain was prominent and immediately incubated in eosin for 2 minutes. Excess eosin was washed off the slides. Slides were then serially dehydrated using ethanol and mounted with Permount mounting media. Cover slips were used to cover the mounted, stained sections and imaged using the automated Lionheart LX.

### Statistical Methods

The data is presented as the means±standard deviations of the experiments (n=3-5). Statistical analysis was performed via one-way ANOVA with posthoc Tukey's test using GraphPad Prism (v 8.2.1).

### Example 2

### Synthesis and Characterization of Electronically Active Defect-Rich Nanoassemblies

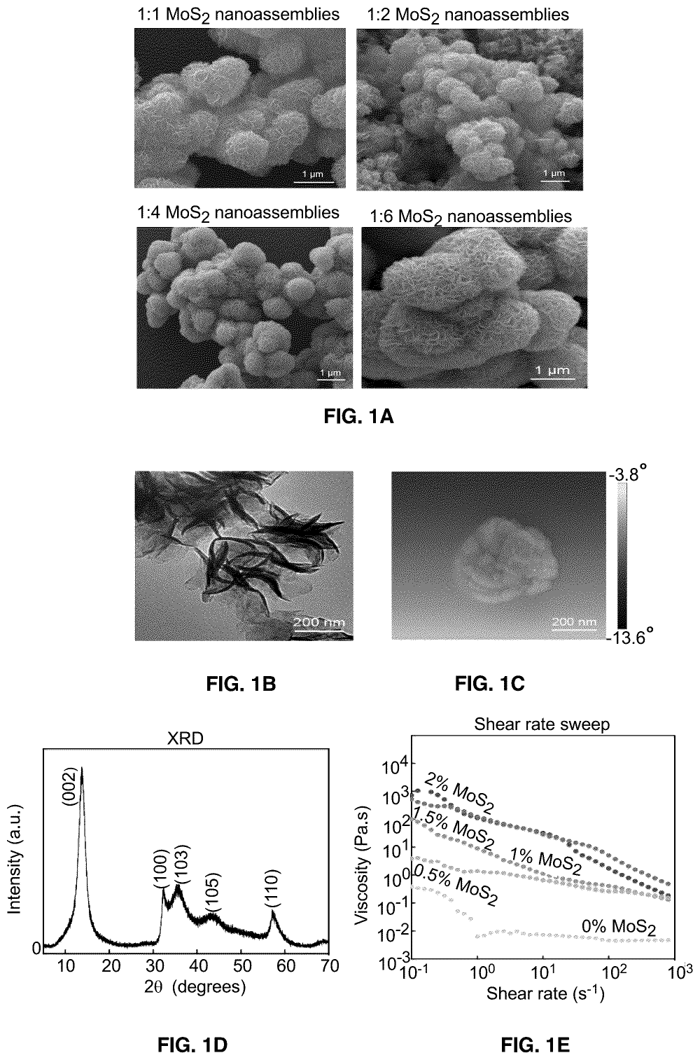

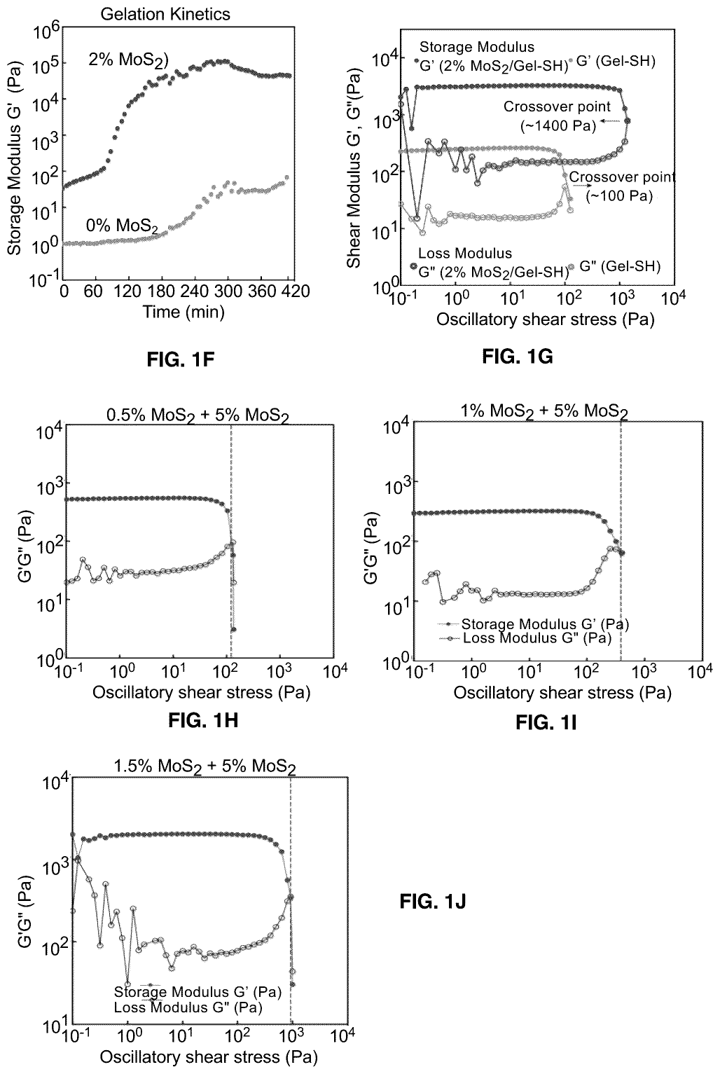

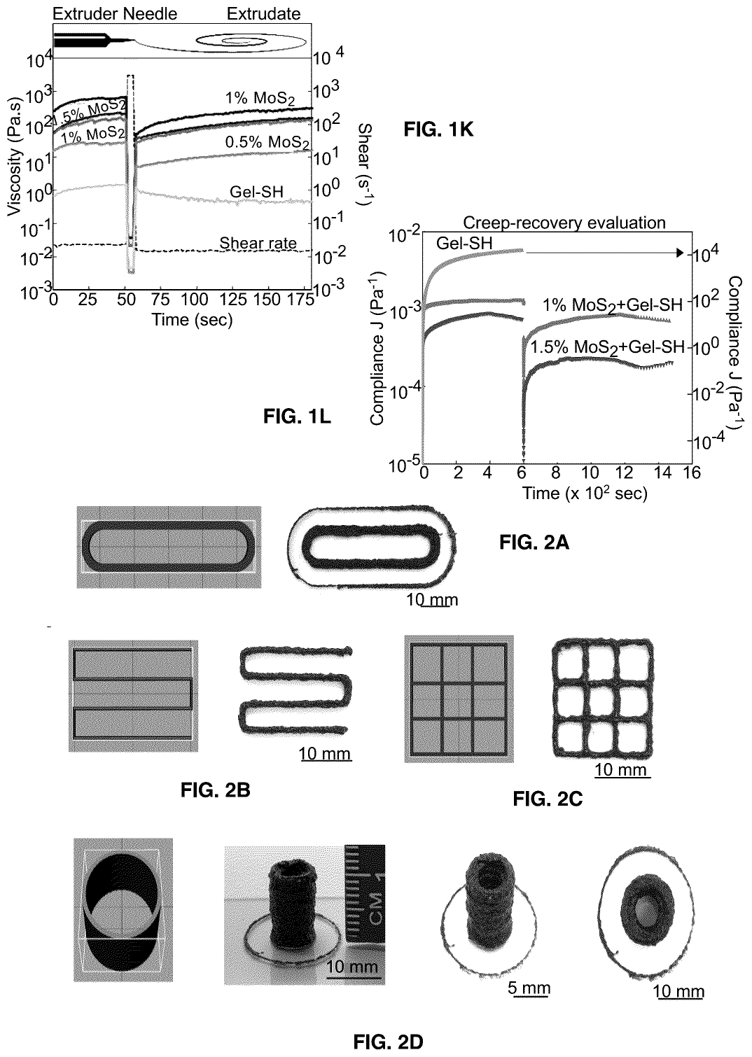

Defect-rich MoS₂ nanoassemblies were synthesized via a hydrothermal route by modulating the ratio of sulfur and molybdenum precursors, similar to our previous work (28). Ratio of precursors of molybdenum (hexaammonium heptamolybdate): sulfur (thiourea) were varied from 1:1 through 1:6 during the synthesis to modulate defect density. Molybdenum resides in a trigonal prismatic coordination (Mo^{IV}), and is bound to six Sulphur atoms, whereas Sulphur (S²⁻) resides in a pyramidal location and is bound to six molybdenum atom (29). This coordination geometry results in formation of a layered structure with linked trigonal prisms, resulting in layers of Sulphur atoms sandwiching molybdenum atoms. The MoS₂ nanosheets thus formed appear to be a hierarchical assembly of well-segregated and concentric architecture of 2D nanosheets resembling a flower morphology. MoS₂ nanoassemblies were examined by scanning electron microscopy (SEM) and agglomerates exhibited a size of ˜1 μm (FIG. 1A). MoS₂ nanoassemblies with various defect densities were also synthesized and examined through SEM (FIG. 1A). The transmission electron microscopy (TEM) (FIG. 11B) and atomic force microscopy (AFM) imaging (FIG. 1C) of dispersed MoS₂ nanoassemblies corroborated its rippled nanoflake morphology, which prevents flake stacking, thus confirming the potential for easy access to active defect centers. Additionally, X-ray diffraction (XRD) and X-ray photoelectron spectroscopy (XPS) were performed on the 2D MoS₂ nanoflower assemblies to validate its hexagonal 2H phase arrangement. The diffraction pattern peaks (FIG. 1D) of MoS₂ nanoassemblies (002, 100, 103, 105 and 110) concur with the hexagonal MoS₂ (JCPDS card No. 73-1508) suggesting high purity of the synthesized nanoflower assemblies. The Scherrer equation was used to calculate crystallite size and it was found to be ˜5.26 nm. XPS characterization of the 1:6 MoS₂ nanosheets was performed and peaks for Mo and S were deconvoluted. The binding energies for molybdenum core level 3d; Mo⁴⁺3d_{5/2} and 3d_{3/2} were 227.5 and 230.8 eV, respectively, while the doublet for sulfur S²⁻ 2p_{3/2} and 2p_{1/2} were recorded at 160.4 and 161.5 eV, respectively. The difference between binding energies of Mo⁴+3d_{5/2} and 3d_{3/2} is 3.3 eV indicated the presence of dominant 2H phase. These investigations confirmed chemical composition of the MoS₂ remained unaltered and the formation 2H phase in the MoS₂ nanoassemblies.

Defect-Rich Nanoassemblies Conceive Robust Hydrogels without External Stimulant

The 1:6 defect-rich 2D MoS₂ nanoflower assemblies are utilized in this work since they provide highest defect-density. The MoS₂ nanoassemblies present active centers for preferential binding of thiolated gelatin forming a robust covalently crosslinked network which is highly elastomeric. By regulating the concentration of the MoS₂ nanoassemblies various properties of the hydrogel network, such as mechanical stiffness, gelation kinetics, electronic and physiochemical characteristics, can be modulated. These hydrogels crosslink spontaneously, without requiring any external stimulus such as chemical, or thermal energy, or UV irradiation and hence do not introduce any extraneous hazardous and harmful impact to the living tissues. Due to the abundance of defect sites, the 1:6 MoS₂ nanoassemblies are expected to actively react with 5 wt % Gel-SH. Additionally, key flow properties of the conductive polymer precursors, which comprised the nanocomposite hydrogel at various MoS₂ concentrations (0.5-2 wt %), were also investigated to achieve optimum rheological properties.

MoS₂ nanoassemblies modulate rheological Response

The viscoelastic measurements of the prepolymer solution, (5 wt % thiolated gelatin, Gel-SH) was investigated at various MoS₂ concentrations by fitting the power law model, η=Kγⁿ⁻¹ to rheological shear-rate sweeps, where η is the viscosity, K is the flow consistency index, γ is shear rate and n is the shear-thinning index (Table 1, FIG. 1E).

TABLE 1
η = K\dot{γ}ⁿ⁻¹

| | K | n |
|---|---|---|
| Gel-SH | 0.0048 | 1.0 |
| 0.5% MoS₂ | 0.84 | 0.72 |
| 1% MoS₂ | 2.41 | 0.53 |
| 1.5% MoS₂ | 4.04 | 0.26 |
| 2% MoS₂ | 101.27 | 0.15 |

For a shear-thinning fluid 0<n<1 while Newtonian fluids have n=1. Close examination revealed Newtonian behavior of the control sample (0 wt % MoS₂) between the shear rates of 0.1-1000 s⁻¹ which, upon addition of MoS₂, exhibited enhanced shear-thinning characteristics (FIG. 1E). For instance, the initial viscosity of 2 wt % MoS₂/Gel-SH was ˜1,000 Pa·s which was drastically reduced to less than 1 Pa·s at shear rate of 1,000 s⁻¹ (n=0.15). Similarly, a corresponding shear-thinning behavior was exhibited in Gel-SH with 1.5, 1 and 0.5 wt % MoS₂ concentrations (n=0.26, 0.53 and 0.72, respectively) as the shear rate increased from 0.1 to 1,000 s⁻¹.

Further, the addition of MoS₂ nanoassemblies to Gel-SH lead to defect-driven gelation which was characterized by monitoring the gelation kinetics of the system (FIG. 1F). The polymer precursors with 5 wt % Gel-SH and 2 wt % MoS₂ nanoassemblies were placed in deionized (DI) water while the storage modulus (G′) of the system was continuously monitored. The storage modulus of the Gel-SH (0% MoS₂, control) did not change substantially over the course of 420 min, however the G′ for 2% MoS₂/Gel-SH significantly increased from its initial value of about 50 Pa to 100 kPa in approximately 270 min and attained the plateau. This indicated the formation of viscoelastic hydrogel due to the interactions between defect rich MoS₂ and the thiolated gelatin hydrogel network, the result of covalent bond formation.

An oscillatory stress-sweep experiment was conducted to further understand the interactions of polymer with progressive concentration of MoS₂ in the hydrogel precursors (FIG. 1G). The cross-over point between storage modulus (G′) and loss modulus (G″) indicates yielding and was =100 Pa for Gel-SH control which was enhanced to =1,400 Pa for 2 wt % MoS₂/Gel-SH. Similarly, for 1 wt % & 1.5 wt % MoS₂/Gel-SH groups the yield was also enhanced, indicating stronger interactions, and occurred at 400 Pa and 1,000 Pa, respectively (FIG. 1H-1J), illustrating significant reinforcement with increased concentration of MoS₂ nanoflower assemblies. A peak-hold test was carried out on the hydrogel precursors to simulate 3D printability through rheological analysis (FIG. 1K). Rheometry was utilized to observe the change in viscosity with a change in shear rate. During initial stages, the printing dope or ink is in the barrel and hence experiences low shear rate (γ<0.1 s⁻¹). The printing ink then enters the needle where shear-rates increase to 100-1,000 s⁻¹, this corresponding high shear-rate should shear-thin the ink. Upon exiting the needle, the flow stops and the biomaterial inks should regain their viscosities, close to 80% of their initial values. The MoS₂/Gel-SH precursors demonstrated regained viscosities in the region simulating recovery, indicating they can be used for 3D printing.

The effect of MoS₂ nanoassemblies on the viscoelastic properties of Gel-SH precursor solution was also examined through creep experiments. The creep-compliance curves of the samples were fitted with Burger model to obtain values of J₀, J₁, τ, and η (Table 2, FIG. 1L).

TABLE 2
J_{c}(t) = J₀ + J1[1 + exp(−t/τ)] + 1/η

| | Gel-SH | 1% MoS₂ | 1.5% MoS₂ |
|---|---|---|---|
| J₀ (Pa⁻¹) | 1.1 × 10⁻⁴ | 1.8 × 10⁻⁴ | 1.2 × 10⁻⁴ |
| J₁ (Pa⁻¹) | 2.0 × 10³ | 9.9 × 10⁻⁴ | 4.6 × 10⁻⁴ |
| η (Pa · s) | 3.3 × 10⁻² | 2.0 × 10⁶ | 1.1 × 10⁶ |
| τ(t) | 257.91 | 0.87 | 0.03 |

The compliance curves highlight recovery for samples with MoS₂ nanoassemblies while only Gel-SH samples (0 wt %, control) illustrated high compliance but no recovery (FIG. 1L). Compliance is inversely proportional to modulus and the overall results further indicate MoS₂ nanoassemblies bolstering the Gel-SH network.

### Nanoengineered Ink Demonstrates 3D Printability

To demonstrate 3D printability, extensive 3D printability assays were performed with the nanoengineered ink. The ability to form continuous filament and exhibit uniform extrusion is directly proportional to hydrogel precursor's shear-thinning and recovery characteristics. First a hanging filament quantification was performed and it was found the extruded filament exhibited a hanging length of 37.5 mm. This verified that the ink is extrudable uniformly into filaments demonstrating shear-thinning and shear-recovery of the ink, for printing multi-layer constructs. To evaluate simple printability various basic shapes were 3D printed. A circular band, rectangular curved lines, and a cross hatch grid pattern were successfully printed (FIG. 2A-2C). A hollow cylinder to a height of 12 mm was successfully 3D printed thus demonstrating the intra-layer stability of the hydrogel ink (FIG. 2D). These 3D printings demonstrate the printability of the ink and showcases its versatility.

### Defect Engineered MoS₂ Nanoassemblies Allows for Covalent Crosslinking

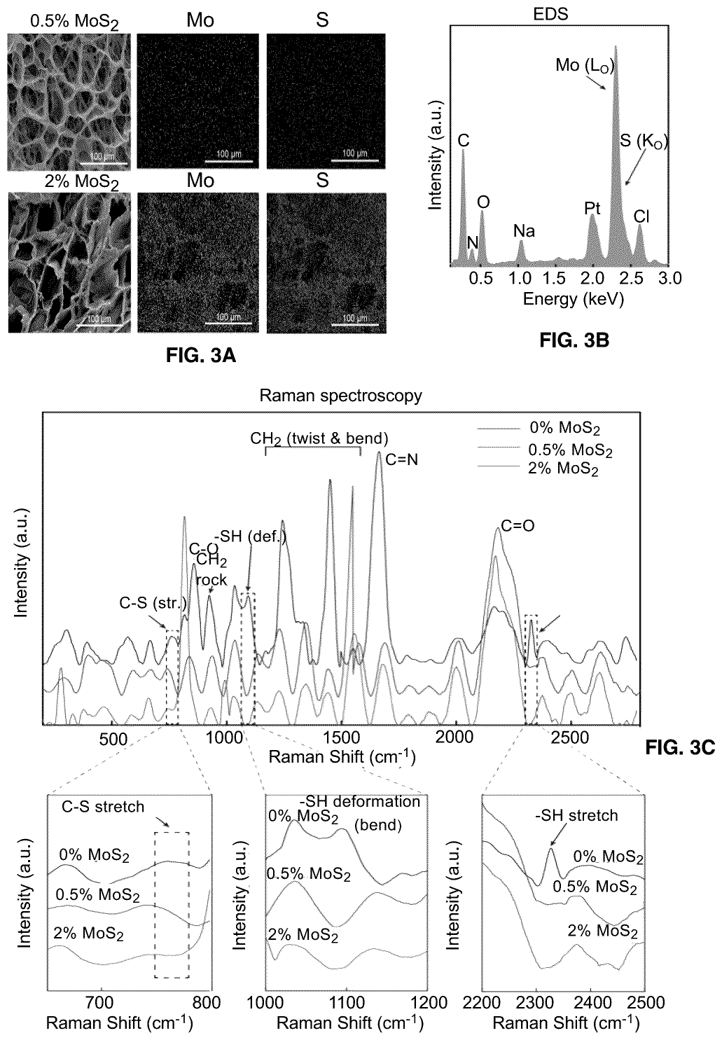

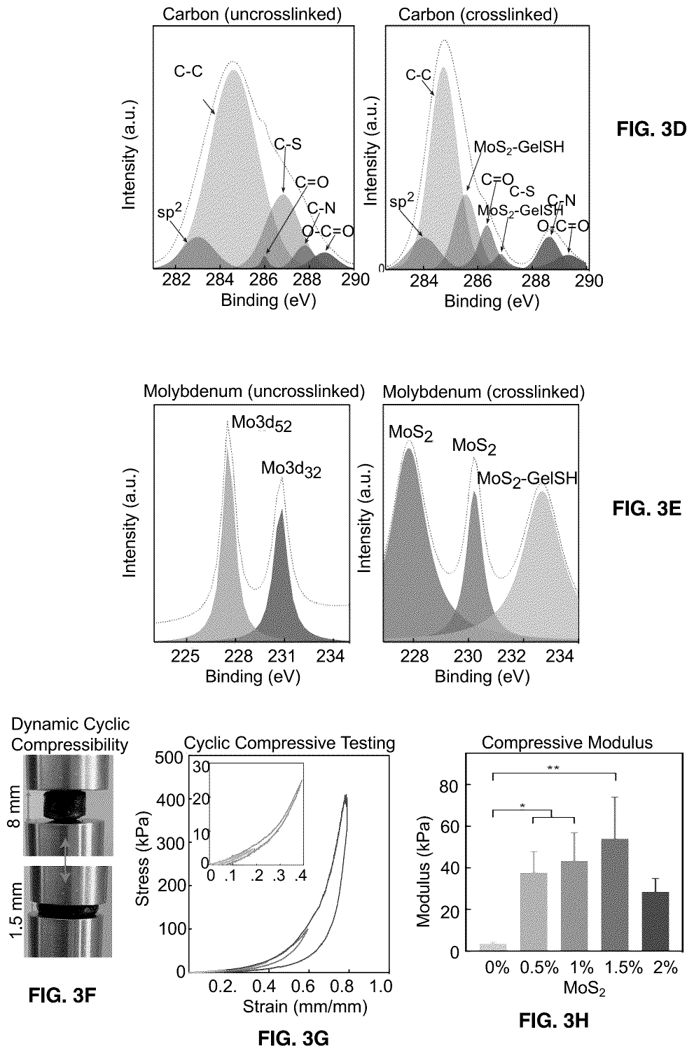

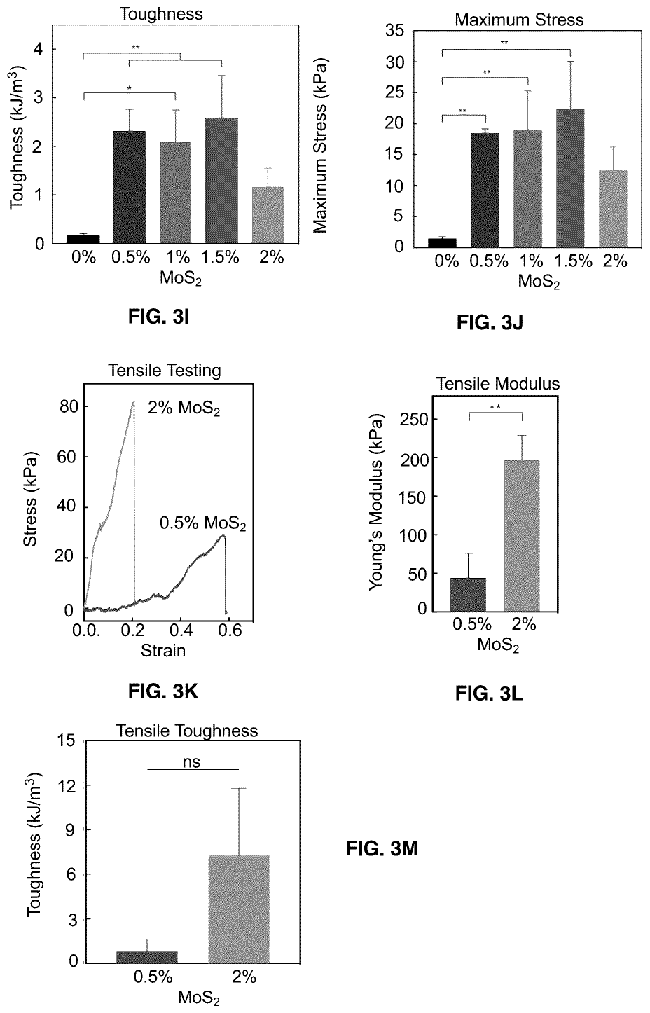

The elastomeric characteristics, flexibility and robustness of the crosslinked networks were demonstrated through simple maneuvers, for example, manual or finger compression. The microstructural characterization, of the crosslinked hydrogel network using scanning electron microscopy (SEM) with energy-dispersive X-ray spectroscopy (EDS) mapping was carried out on the transverse sections of freeze-dried hydrogel samples. SEM imaging on the transverse cross-sections (FIG. 3A) reveal interconnected porous hydrogel network wherein for 0.5% MoS₂/Gel-SH sample the pore size was ≈50 μm which was enhanced to ≈100 μm for 2 wt % MoS₂/Gel-SH sample. Additionally, through EDS image mapping (FIG. 3A) it was determined that Mo and S atoms were distributed across the crosslinked hydrogel network with a higher Mo and S concentration in the 2 wt % MoS₂/Gel-SH sample. The presence of Mo and S in the samples was also validated through EDS spectra of the hydrogels (FIG. 3B). The increase in hydrogel pore size could be attributed to enhanced interactions between MoS₂ nanoassemblies and Gel-SH which could locally condense the polymer fraction to result in the formation of larger voids.

Molybdenum's natural affinity to thiol chemistry allows the covalent crosslinking of the polymer network with defect-rich MoS₂ nanoassemblies where the vacancy centers are mostly due to missing sulfur atoms in the lattice structure. To validate this hypothesis, Raman spectroscopy was performed for Gel-SH and chemically cross-linked MoS₂ hydrogel samples (FIG. 3C). Raman spectra for control (Gel-SH) exhibited two characteristic peaks at around 1099 and 2330 cm¹ which can be assigned to thiol deformation and thiol stretching, respectively. The intensities of these two peaks reduced substantially with the addition of MoS₂ samples. Additionally, there was a peak shift and attenuation of C—S stretching observed with addition of MoS₂ in Gel-SH at 760 cm⁻¹ due to covalent crosslinking between the thiol group and MoS₂. Overall, the attenuations in Gel-SH vibrations with addition of MoS₂ indicated the presence of MoS₂ nanoflower assemblies as crosslinking centers.

XPS characterization was also performed to substantiate Raman spectroscopy data on ascertaining crosslinking mechanism. XPS analysis of electrons from carbon (1s) and molybdenum (3d) core levels was performed for control Gel-SH and 0.5 wt % MoS₂/Gel-SH crosslinked hydrogel samples. In carbon (1s) XPS spectra of Gel-SH, the binding energy (B.E.) peaks deconvoluted at 283, 284.6, 286.1, 286.8, 287.8 and 288.7 eV can be assigned to sp², C—C, C═O, C—S, C—N and O—C═O chemical states respectively (FIG. 3D). After chemical crosslinking with MoS₂ nanoassemblies, the corresponding B.E. decreased, mainly due to formation of C—S—Mo in the hydrogels. In molybdenum (3d) XPS spectra, peaks belonging to Mo 3d_{5/2} and Mo 3d₃₁₂ were observed at 227.5 and 230.8 eV, respectively (FIG. 3E). After crosslinking, the binding energy of these peaks shifted to 227.8 and 230.2 eV, respectively, in addition to new peak was observed at 232.7 eV due to C—S—Mo chemical state. Both Raman and XPS analysis confirmed the formation of covalent crosslinking through defect-driven gelation between Gel-SH and MoS₂ nanoassemblies.

### MoS₂ Nanoassemblies Regulate Elastomeric Characteristics of the Nanoengineered Hydrogels

The elastomeric nature of the crosslinked hydrogels was further characterized by uniaxial compressive testing (FIG. 3F). The flexible implanted devices often undergo dynamic stress and therefore must have the ability to withstand without compromising their elastomeric nature. To assess this, the composite hydrogels were further subjected to cyclic compressive tests wherein 1.5 wt % MoS₂/Gel-SH hydrogel system was made to undergo five continuous compression cycles varying from 20% to 80% strain (FIG. 3G). The samples showed almost complete recovery in each cycle. The room temperature compressive modulus (FIG. 3H), toughness (FIG. 3I) and maximum stress (FIG. 3J) values were calculated from stress-strain curves of the various hydrogel systems. The 5 wt % Gel-SH was dissolved in DI water and thermally gelled at 37° C. to be used as a control. The Gel-SH control displayed a compressive modulus of ≈4 kPa which increased to ≈38, 44 and 55 kPa for 0.5 wt %, 1 wt % & 1.5 wt % MoS₂/Gel-SH, respectively. A further increase in MoS₂ concentration to 2 wt % MoS₂/Gel-SH resulted in slight decline in the compressive modulus which can be primarily due to enhanced interactions between MoS₂ nanoassemblies and Gel-SH, resulting in larger network voids and excess MoS₂ could be acting as network defects.

Next, elongation tensile testing of hydrogel groups was performed to comprehend its tensile properties. In the tensile stress-strain curve (FIG. 3K) it was observed 0.5% MoS₂/Gel-SH hydrogels were able to elongate up to 60% strain and thus, were more flexible, justifying its use in flexible electronics. While 2% MoS₂/Gel-SH did not demonstrate significant elongation and fractured at 20% strain. The tensile young's modulus (FIG. 3L) and toughness (FIG. 3M) of the hydrogels was also quantified. 2% MoS₂/Gel-SH hydrogels demonstrated modulus of ≈200 kPa and toughness of ≈7 kJ/m³ which indicates stiff and brittle hydrogel network whereas 0.5% MoS₂/Gel-SH hydrogels had modulus ≈50 kPa and toughness ≈1.5 kJ/m³ indicating more softer, stretchable networks.

Additionally, the torsional deformation of the hydrogels was examined through oscillatory rheology on the crosslinked hydrogels. Firstly, stress relaxation characterization was performed where we increased the strain on the hydrogels was increased by 5% every 250 secs till 20% strain and analyzed variation in its storage modulus. Both 0.5% and 2% MoS₂ hydrogels demonstrated a stable storage modulus over the entire test period of 1000 secs without any network relaxation. Next, we performed oscillatory stress sweeps on both hydrogels and reported higher crossover yield point for 0.5% MoS₂ as compared to 2% MoS₂. This was also reflected in its corresponding yield stress quantification in which yield stress for 0.5% MoS₂ was 14 times higher at ≈620 Pa than 2% MoS₂ at ≈50 Pa. The higher yield point indicates stronger hydrogel network bonding. Overall, these results established the elasticity, mechanical integrity, and robustness of the MoS₂/Gel-SH hydrogels, illustrating a strong interaction and chemical conjugation between the MoS₂ nanoassemblies and Gel-SH. They also help in better understanding the deformation mechanics of these hydrogels, aiding in designing flexible bioelectronics with improved performance.

### Nanoengineered Hydrogels Demonstrate Physiological Stability

The hydrolytic and enzymatic swelling and degradation characteristics of the crosslinked MoS₂ hydrogels were also examined under physiological conditions. Hydrogels similar to previous studies with varied amount of MoS₂ nanoassemblies (0.5, 1, 1.5 & 2 wt %) were fashioned and thermally gelled thiolated gelatin samples were used as controls. Different batches of hydrogel samples were immersed in PBS and 5 wt % collagenase separately. Collagenase is a proteinase that cleaves the peptide bonds of gelatin. The un-crosslinked control samples without MoS₂ swelled to almost twice its weight in 3 days, but soon afterwards degraded completely through day 7 under hydrolytic conditions (buffer) whereas under collagenase degradation the samples completely degraded in 36 hours. Contrarily, the crosslinked MoS₂/Gel-SH hydrogels remained stable for 21 days in both hydrolytic and enzymatic conditions. Under hydrolytic conditions 0.5 wt % MoS₂/Gel-SH hydrogels swelled to approx. 1.5 times after 7 days, after which its weight was reduced to its original value. No substantial swelling was observed in 1, 1.5 & 2 wt % MoS₂/Gel-SH hydrogels for 21 days. This is mainly due to the fact that with increased content of MoS₂ nanoassemblies, the excess MoS₂ resides in the hydrogel pores and prevents substantial water uptake. Under enzymatic conditions 0.5, 1 & 1.5 wt % MoS₂/Gel-SH hydrogels exhibit gradual swelling to 1.2-1.6 times their original weight in 36 hours whereas 2 wt % MoS₂/Gel-SH hydrogels exhibited more or less no swelling. All the hydrogel samples did not degrade and remain stable for 21 days even in enzymatic conditions. Overall, the results indicate the crosslinked hydrogels would remain stable under hydrolytic and enzymatic conditions for a prolonged period which demonstrates their applicability as potential tunable implantable biointerfaces.

### Electronically Active MoS₂ Regulate Electrical and Electrochemical Performance of Nanoengineered Hydrogels

For electrical and electrochemical investigation of the samples, MoS₂ and MoS₂/Gel-SH were pressed into pellets and their conductivity was measured (n=3) via the 4-point probe method. The pure MoS₂ had a sheet resistance of 52±16 Ω/sq which confirmed the successful synthesis of conductive MoS₂ nanoassemblies. The sheet resistance of 10 wt % MoS₂/Gel-SH was measured to be 0.16±0.26 MΩ/sq. For those compositions with lower MoS₂ content, the resistance fell out of the range of the multimeter (>120 MΩ). The crosslinked conductive hydrogels were also successfully utilized to make a closed electrical circuit. The 0.5 wt % MoS₂/GeISH hydrogels were serially connected to a LED bulb, 1 kΩ resistance and subsequently to a DC power supply. The hydrogels were able to conduct the electric current and make the LED bulb glow. The hydrogel precursors were also 3D printed and subsequently connected in the same circuit which also caused the LED to glow. This shows significant potential of utilizing these conductive hydrogel precursors for 3D printing soft actuators, tissue-engineered grafts and sensoric patches.

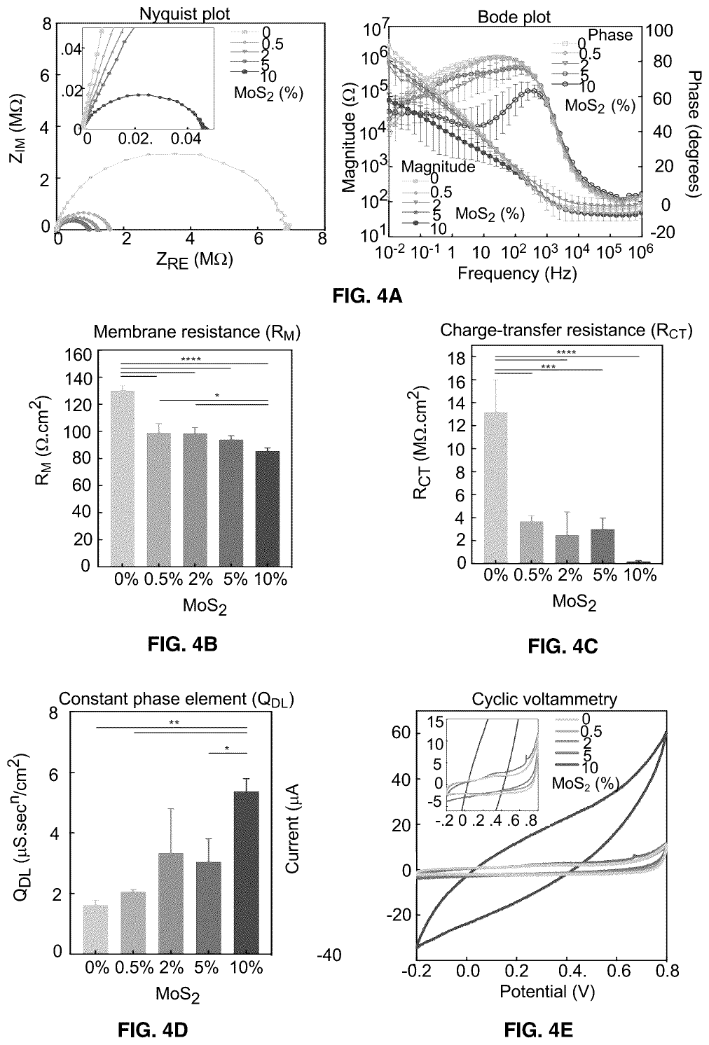

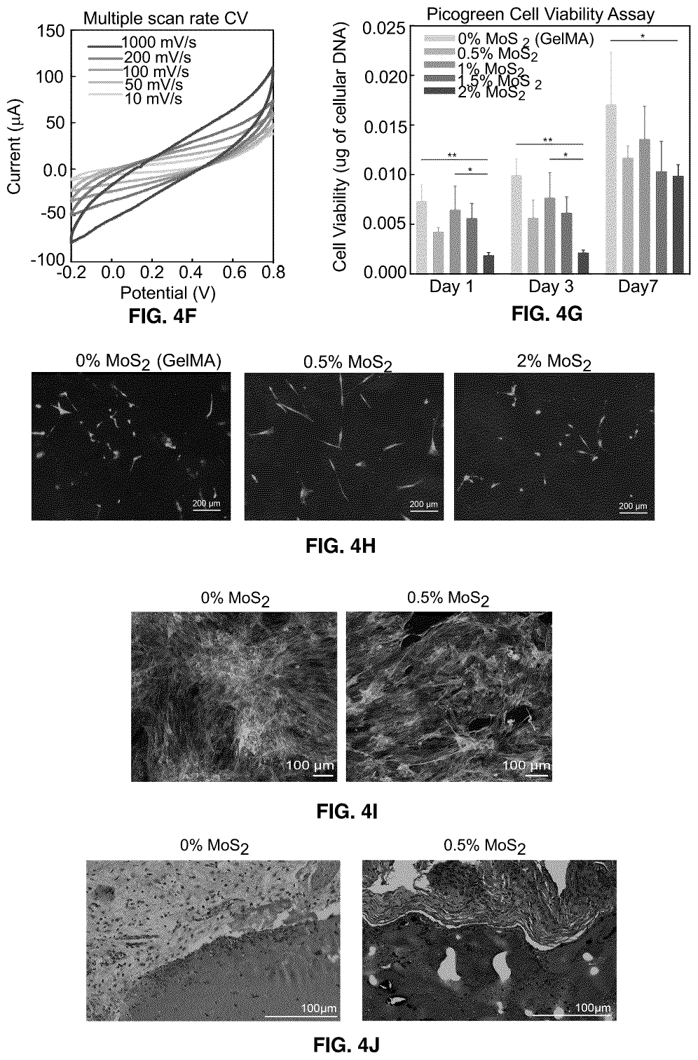

Electrochemical characterization of MoS₂/Gel-SH hydrogels was performed at room temperature using electrical impedance spectroscopy (EIS). A subsequent equivalent circuit analysis (FIG. 4A) was performed via multiple scan rate cyclic voltammetry (CV) in 0.1 M PBS 7.4 as electrolyte. The membrane resistance, R_{M} (FIG. 4B), of thermally gelled Gel-SH control was found to be 131±3.3 Ω·cm² which dropped slightly to 99.1±6.6 Ω·cm² and 85.5±2.4 Ω·cm² upon inclusion of 0.5 wt % and 10 wt % MoS₂, respectively. The hydrating PBS buffer was conductive enough (R_{S}=174±6.6 Ω·cm²) to overshadow the hydrogel, hence while MoS₂ crosslinked hydrogels had lower R_{M}, the change was not dramatic.

The presence of MoS₂ as a crosslinker of the thiolated-gelatin affected the charge transfer resistance (R_{CT}) significantly (FIG. 4C). The R_{CT} of Gel-SH (negative control) was 13.2±2.8 MΩ·cm², reflecting the absence of any electroactive species, which dropped to 3.69±0.47 MΩ·cm² and 0.16±0.09 MΩ·cm² upon inclusion of 0.5 wt % and 10 wt % MoS₂ concentrations, confirming the electroactivity of MoS₂ and suggesting the MoS₂ vacancies provided a path for charge propagation throughout the hydrogel. The highly porous and swellable gelatin-based hydrogel provided a path for capacitive charging currents, however, addition of electronically conductive MoS₂ introduced a new path for resistive currents within the hydrogel. This indeed was observable in the CVs of hydrogel composites; the semi-rectangular voltammogram, representative of an ideal capacitive behavior of gelatin control, became distorted in the 10 wt % MoS₂/Gel-SH (FIG. 4E) suggesting a pseudocapacitive charge storage behavior resulting from both capacitive and faradic currents (30). This pseudocapacitive response offers both high rate capability and high capacitance, which can be successfully utilized for designing 2D nanomaterial-based energy storage material systems (31).

The constant phase element (CPE) of the Randles Equivalent Circuit, which is representative of a non-ideal double layer capacitor, had a three-fold increase upon inclusion of 10 wt % MoS₂/Gel-SH compared to the Gel-SH control hydrogel (FIG. 4D). The value of the CPE exponent, n, decreased upon inclusion of 10 wt % MoS₂/Gel-SH, indicating further deviation from an ideal capacitor. The CPE represents an ideal capacitor when n equals 1. Values lower than 1 are a measure of the extent of the non-ideality of the capacitor. The higher double layer capacitance of 10 wt % MoS₂/Gel-SH was confirmed by voltammograms as well. The hysteresis envelope of CVs appeared as a result of non-Faradaic current which governed the magnitude of double layer capacitance (C_{dl}). Multiple scan rate cyclic voltammetry (MSRCV) of the Gel-SH control and the 10 wt % MoS₂/Gel-SH (FIG. 4F) at different scan rates showed that the CVs maintained the semi-rectangular shape expected from capacitors. The MoS₂ containing composite hydrogels, as SEM images revealed, have a highly porous structure which is consistent with a capacitive response. Additionally, the polarization resistances, Rp, calculated from the CV over a short range (30 to 40 mV) at a slow scan rate of 10 mV/s, were determined to be 7.00 MΩ·cm², 3.61 MΩ·cm², 1.88 MΩ·cm², 0.59 MΩ·cm², 0.07 MΩ·cm² for the (MoS₂=0, 0.5, 2, 5 and 10 wt %), which were in close agreement with the R_{CT} values calculated from EIS data. In the electrochemical characterization studies, higher concentrations of MoS₂ nanoassemblies was utilized to determine its saturation concentration in the defect-driven crosslinked hydrogels. All remaining studies were performed with lower MoS₂ concentration which fulfills the anatomical requirement

Nanoengineered hydrogel Biointerfaces Demonstrate Favorable Biocompatibility

To assess the biomedical functionality and cellular compatibility of these nanoengineered MoS₂ crosslinked hydrogels, a 2D seeding of human mesenchymal stem cell (hMSC, bone-marrow derived, passage 4) was performed on the printed hydrogels. The cells were able to successfully attach to the surfaces of the hydrogel with different MoS₂ concentration varying from 0.5 wt % to 2 wt %. The cell cytotoxicity and viability were successfully evaluated using a picogreen dsDNA quantification assay (FIG. 4G). Since, Gel-SH is thermally gelled and dissolves at 37° C., a photocrosslinked GeIMA sample was used as positive control. It was found that cell-viability increased gradually from day 1 to day 7 in hydrogels with 0.5 wt %, 1 wt % and 1.5 wt % MoS₂ samples, similar to control. The μg of cellular DNA was initially low in the case of hydrogels with 2 wt % MoS₂ for day 1 and day 3, but there was almost 78% increase in μg of cellular DNA at day 7. Thus, the crosslinked MoS₂ hydrogels are cytocompatible and viable for cellular proliferation. Live/dead imaging using epifluorescence microscopy also revealed high cell viability over 72 hours on the crosslinked hydrogels indicated by the high number of live (green) cells compared to dead (red) cells (FIG. 4H).

To understand the cellular morphology and spreading of the hMSCs on the hydrogels, cell cytoskeletal staining was performed (FIG. 4I). It was found the cells were able to successfully attach and spread on the hydrogel surface for all groups, as compared to control and exhibited spindle shape morphology. This shows the potential of the hydrogels to be used as cellular constructs for various applications in regenerative medicine and tissue engineering.

To discern the potential of the developed hydrogel for various clinical applications, it is important to study the in-vivo biocompatibility of the developed MoS₂/Gel-SH hydrogels. Similar to previous study, GeIMA hydrogel was used as control as Gel-SH would disintegrate at 37° C. and in vivo studies with GeIMA have been well established (32). 0.5 wt % MoS₂/Gel-SH and GeIMA hydrogels were implanted subcutaneously and were surgically extracted from the dorsal pocket of male C57BL/6J mice after 7 days. While extracting the implant, we observed that the implant did not displace from the site of implantation and was free of infection. H&E staining of explanted tissue can be seen showing GeIMA hydrogel and 0.5 wt % MoS₂/Gel-SH hydrogel (FIG. 4J), highlighting the surrounding and infiltrated tissue. Gross examination of the implanted hydrogels showed no necrosis. Additionally, the inflammation produced around the GeISH-0.5% MoS₂ hydrogel was comparable to the GeIMA control (33,34). Thus, we elucidate that the host-body response was equivalent to GeIMA. These in vivo results, in addition to the in vitro findings, illustrate potential of these nanoengineered, mechanically tough, and conductive nanobiocomposite hydrogels may be further studied as in vivo implants for diverse biomedical applications such as in-situ tissue engineering and regenerative medicine.

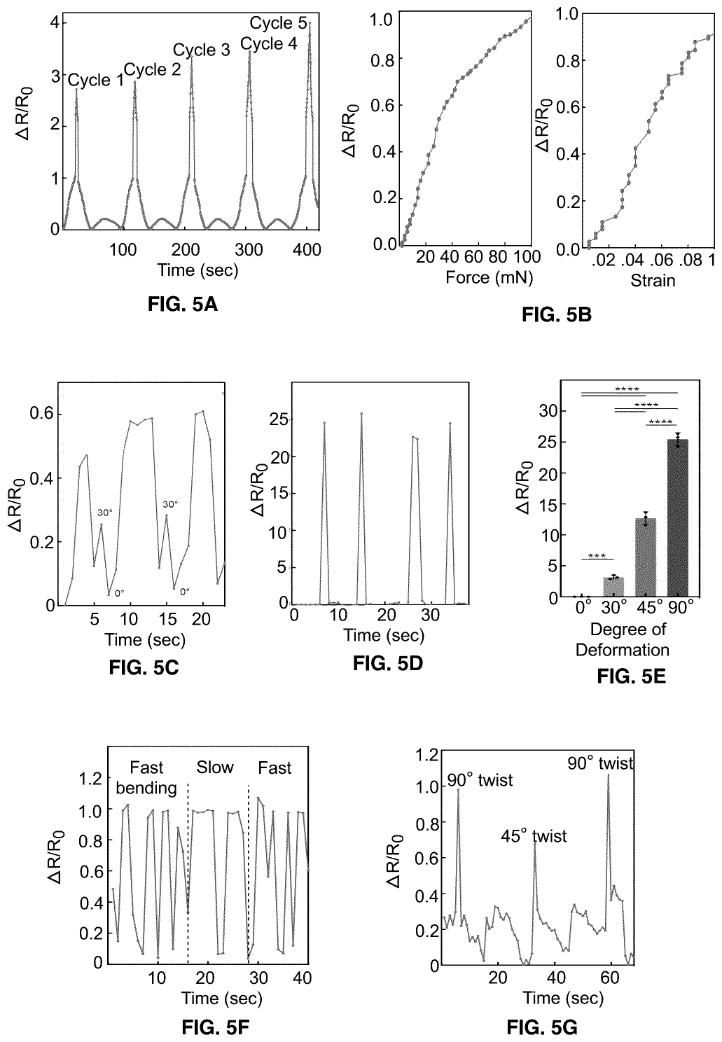

Nanoengineered hydrogels Precisely Sense Dynamic Strains Recently, there has been heightened interest in designing conductive biomaterials for wearable electronics to monitor motions and pressure linked events (35). But the deformation dependent reduction in electrical performance together with low sensitivity, poor signal output and device bending induced failure impedes deployment of conductive hydrogels for variety of different applications. To overcome many of such limitations, we conceived these 3D printable flexible hydrogel sensors that demonstrates high-strain sensitivity with superior accuracy during various deformations and flexed configurations. The resistance changes of the 0.5 wt % MoS₂/Gel-SH hydrogel was evaluated under cyclic compression (FIG. 5A). Each loading and unloading cycle could be mapped with corresponding change in resistance indicating the elastomeric hydrogel to be sensitive under external strain.

The linearity of the sensing platform is crucial in detection of external stimuli. The compression testing demonstrated a direct relationship between force/strain with change in resistance (FIG. 5B). Next as a proof-of-concept wearable device were enclosed between PDMS layers and monitored change in resistance while performing various motions such as bending and twisting. Interestingly, the change in bending angle, had an effect on the change in resistance. For example, smaller (30°) and larger (45°) deformation was identified during a continuous bending cycle (FIG. 5C). Then, after performing continuous multiple double 90° bends, a ˜25-fold change in resistance was a surprising result (FIG. 5D). When different degrees of bending were compared, we could observe highest resistance change for 90° deformation and smallest for 30° deformation as expected (FIG. 5E), accurately capturing deformation sensitive movements. Subsequently we performed single side bending of the hydrogel device with different movement velocities (fast/slow) and were able to capture distinct rates of resistance changes (FIG. 5F). Finally, a twisting action on the hydrogel device was performed and specific resistance change signatures for 90° and 45° twists (FIG. 5G) was captured. It is important to note that all the observed strain-sensitivity is fully reversible thereby paving a path for continuous monitoring. Thus, the developed elastomeric flexible bioelectronics can accurately capture various human motion dynamics and patterns.

The following references are cited herein:
1. Choi et al. Advanced Materials 2016, 28(22):4203-4218.
2. Sekitani et al. Advanced Materials 2010, 22(20):2228-2246.
3. Schwartz et al. Nature communications 2013, 4(1):1-8.
4. Lee et al. Advanced Functional Materials 2019, 29(24):1808075.
5. Kim et al. Nature materials 2010, 9 (11):929-937.
6. Jeerapan et al. Advanced Functional Materials 2019, 1906243.
7. Viventi et al. Science translational medicine 2010, 2(24):24ra22-24ra22.
8. Jeong et al. Neuron 2015, 86(1):175-186.
9. Rivnay et al. Science Advances 2017, 3(6):e1601649.
10. Lacour et al. Nature Reviews Materials 2016, 1(10):1-14.
11. Song et al. Acs Nano 2019, 13(11):12280-12286.
12. Someya et al. Nature 2016, 540(7633):379-385.
13. Yang et al. Nano Research 2017, 10(5):1560-1583.
14. Choi et al. Accounts of Chemical Research 2019, 52(1):73-81.
15. Walther et al. Materials Today 2020, 39:23-46.
16. Chou et al. Nature communications 2015, 6:8311.
17. Wang et al. Advanced materials 2015, 27(44):7117-7122.
18. Nicolosi et al. Science 2013, 340(6139):1226419.
19. Nguyen et al. Advanced Materials 2015, 27(40):6225-6229.
20. Sen et al. ACS applied materials & interfaces 2013, 5(4):1240-1247.
21. Maitra et al. Angewandte Chemie International Edition 2013, 52(49):13057-13061.
22. Pumera et al. TrAC Trends in Analytical Chemistry 2014, 61:49-53.
23. Li et al. ACS applied materials & interfaces 2017, 9(18):15286-15296.
24. Liu et al. Advanced materials 2014, 26(21):3433-3440.
25. Zhang et al. Nature Reviews Methods Primers 2021, 1(1):75.
26. Shin et al. Advanced Materials 2016, 28(17):3280-3289.
27. Gungor-Ozkerim et al, Biomaterials Science 2018, 6(5):915-946.
28. Jaiswal et al. Using Defect-Rich Nanoassemblies of 2D Transition Metal Dichalcogenides and Polymeric Binder for Biomedical Applications. 2017, [0100]29(36):1702037.
29. Li et al. Journal of Materiomics 2015, 1(1):33-44.
30. Yang et al. Composites Science and Technology 2015, 121:123-128.
31. Jiang et al. ENERGY & ENVIRONMENTAL MATERIALS 2019, 2(1):30-37.
32. Noshadi et al. Biomaterials Science 2017, 5(10):2093-2105.
33. Hong et al. Nature Communications 2019, 10(1):2060.
34. Kong et al. Nature Communications 2020, 11(1):1435.
35. Chen et al. npj Flexible Electronics 2020, 4(1):2.
36. Kommareddy et al. Bioconjugate Chemistry 2005, 16(6):1423-1432.
37. Peak, et al. Langmuir 2018, 34(3):917-925.

What is claimed is:

1. A hydrogel construct, comprising:
a 2D MoS₂ nanoassembly crosslinked with a natural polymer.

2. The hydrogel construct of claim 1, wherein the 2D MoS₂ nanoassembly is defect dense.

3. The hydrogel construct of claim 2, wherein the 2D MoS₂ nanoassembly comprises a defect ratio of about 1:0 to about 1:10 molybdenum:sulfur.

4. The hydrogel construct of claim 1, wherein the natural polymer is gelatin, hyaluronic acid, pectin, collagen, carrageenan, chitosan, cellulose, silk fibroin, keratin, alginate, elastin, glucomannan, fibrinogen, gelatin, lignin, starch, xanthan gum, zein, poly-γ-glutamic acid (γ-PGA) or pullulan.

5. The hydrogel construct of claim 4, wherein the natural polymer is thiolated.

6. The hydrogel construct of claim 1, wherein the hydrogel construct comprises a crosslinked gelatin-SH-2D-MoS₂ nanoassembly.

7. The hydrogel construct of claim 1, wherein the hydrogel construct is a 3D printable, biocompatible biomaterial ink.

8. A method for nanoengineering a biomaterial ink, comprising:
synthesizing a defect dense 2D MoS₂ nanoassembly;
thiolating a gelatin to produce a thiolated gelatin; and
crosslinking the 2D MoS₂ nanoassembly with the thiolated gelatin, thereby nanoengineering the biomaterial ink.

9. The method of claim 8, wherein the synthesizing step comprises constructing the 2D MoS₂ nanoassembly with a dense defect ratio of about 1:2 to about 1:10 molybdenum:sulfur.

10. The method of claim 9, wherein the dense defect ratio is 1:6 molybdenum:sulfur.

11. The method of claim 8, wherein the crosslinking step comprises conjugating thiols on the thiolated gelatin in sulfur vacancies within the 2D MoS₂ nanoassembly without external stimuli or an initiator.

12. The method of claim 8, further comprising 3D printing a biocompatible, flexible electronic device from the biomaterial ink.

13. A biocompatible, flexible electronic device nanoengineered by the method of claim 8.

14. The biocompatible, flexible electronic device of claim 13, wherein the 2D MoS₂ nanoassemblies are electronically conductive.

15. The biocompatible, flexible electronic device of claim 13 comprising an electroceutical.

16. The biocompatible, flexible electronic device of claim 13 comprising a wearable device, an ingestable device or an implantable device.

17. The biocompatible, flexible electronic device of claim 13 comprising an energy storage device or a light actuating conductive device.

18. A 3D printed wearable electronic device configured to monitor motion of a subject, comprising:
a 2D-MoS₂ nanoassembly comprising a plurality of sulfur vacancies therein; and
a gelatin comprising a plurality of thiols attached thereto, each of said plurality of thiols crosslinked within one of the plurality of sulfur vacancies.

19. The wearable electronic device of claim 18, wherein the 2D-MoS₂ nanoassembly comprises a defect ratio of 1:2 to about 1:10 molybdenum:sulfur.

20. The wearable electronic device of claim 18, comprising a flexible, biocompatible hydrogel construct.

21. The wearable electronic device of claim 20, wherein the flexible, biocompatible hydrogel construct is an electroceutical.

22. The wearable electronic device of claim 18, comprising an ingestible device.

23. The wearable electronic device of claim 18, comprising hybrid conductivity, charge storage or tissue-like mechanical properties.

24. A method for determining a treatment for a subject in need thereof, comprising:
placing the wearable electronic device of claim 18 on or in the subject;
monitoring at least one dynamic parameter of the subject via the wearable electronic device; and
selecting a treatment for the subject based on results of the monitoring step.

25. The method of claim 24, wherein the wearable electronic device is an electroceutical, said method further comprising delivering the treatment to the subject.

26. The method of claim 24, wherein the dynamic parameter is motion, temperature, humidity, electric current, flow, strain, or pressure or a combination thereof.
